(19) United States
(12) Patent Application Publication
(10) Pub. No.: US 2015/0017266 A1
(43) Pub. Date: Jan. 15, 2015

(54) Compositions and Methods for Relieving Symptoms of Rheumatoid Arthritis and Related Illnesses

(71) Applicant: LUCILLE TOWNSEND, CLEVELAND, TN (US)

(72) Inventor: LUCILLE TOWNSEND, CLEVELAND, TN (US)

(21) Appl. No.: 14/504,418

(22) Filed: Oct. 2, 2014

Related U.S. Application Data
(63) Continuation-in-part of application No. 13/604,424, filed on Sep. 5, 2012.

(51) Int. Cl. A61K 31/59; A61K 33/06; A61K 31/375; A61K 33/00

(52) U.S. Cl. CPC A61K 31/59; A61K 33/00; A61K 33/06; A61K 31/375
USPC 424/682; 424/722

(57) ABSTRACT

Compositions and methods for relieving symptoms of rheumatoid arthritis (RA) and related illnesses are disclosed. It appears that the primary cause of RA is a sensitivity or hypersensitivity to potassium and/or vitamin C, or too much potassium and/or vitamin C in the diet. The effects of potassium and vitamin C on the body must be modified to relieve the symptoms of RA and related illnesses. It appears that magnesium does not cause the inflammation, but appears that it exacerbates the problem. It appears that calcium and vitamin D intake, possibly in combination with the reduced or no intake of potassium and/or vitamin C, may also help to relieve the symptoms of RA and related illnesses.

### CROSS-REFERENCE TO RELATED APPLICATIONS

The present application claims the priority benefit of U.S. Nonprovisional Patent Application Ser. No. 13/604,424, filed on Sep. 5, 2012, which claims the priority benefit of U.S. Provisional Patent Application Serial No. 61/573,546, filed on Sep. 8, 2011, both of which are incorporated herein by reference in their entireties.

### BACKGROUND OF THE INVENTION

### Field of the Invention

The present invention relates to compositions and methods for relieving symptoms of rheumatoid arthritis and related illnesses.

It appears that the primary cause of rheumatoid arthritis (RA) is a sensitivity or hypersensitivity to the mineral potassium and/or vitamin C. When the Applicant was diagnosed with the disease years ago, it became necessary for the Applicant to know what was causing such searing pain and swelling in all of the Applicant's joints. The Applicant has come from being bedridden and crippled having swollen fingers, hands, knees, ankles, and elbows that the Applicant was unable to hold a fork to feed herself. After discovering the present invention and without the use of any medication, the Applicant can write with a pen.

Although potassium can be an important mineral, in some people the mineral may act as a drain stopper causing the body to retain fluids. It is believed that any liquid that is ingested remain in the body instead of leaving the body through normal elimination. Although the body will eliminate some urine, it will be slow and only a small amount. Much like a slow drain in a sink, any additional liquid will force the body to find other places to store the overflow until it gradually recedes. An ultrasound will show normal kidneys but their function is hampered by the mineral potassium. It is like waiting overnight for a pint of water to go down the kitchen sink. The excess fluids take up residence anywhere and everywhere. This may be why the joints of RA patients are deformed. The knuckles and other joints swell as a result of excess fluid around joint causing them to expand and become twisted and deformed. The longer the inflammation remains around the joint the more deformed the bone becomes. It is the inflammation that causes the pain and the deformity. Mystery pains in the arms, shoulders, and back called fibromyalgia are just the result of excess fluids not having anywhere else to go. Even some headaches are the pressure of an overflow of fluid in the brain. The body has nowhere to store it so the body swells inside and out until the kidneys can slowly drain it out.

The Applicant is unaware of inventions or patents, taken either singly or in combination, which are seen to describe the present invention as claimed.

### SUMMARY OF THE INVENTION

The present invention relates to compositions and methods for relieving symptoms of rheumatoid arthritis and related illnesses.

It appears that the primary cause of RA is a sensitivity or hypersensitivity to potassium and/or vitamin C, or too much potassium and/or vitamin C in the diet. The effects of potassium and vitamin C on the body must be modified to relieve the symptoms of RA and related illnesses.

Also, build-up of acid in the body occurs as a result of too much potassium and/or vitamin C. Build-up of acid in the body may be neutralized with calcium and vitamin D (for calcium absorption) or the body will retain fluids in an attempt to dilute said acid. Preferably, a greater amount of vitamin D than the recommended daily amount is needed.

It appears that the mineral magnesium does not cause the inflammation, but appears that it exacerbates the problem.

### DETAILED DESCRIPTION OF THE PRESENT INVENTION

The present invention relates to compositions and methods for relieving symptoms of rheumatoid arthritis (RA) and related illnesses, such as, but not limited to, fibromyalgia, headaches and migraines.

It appears that the primary cause of RA is a sensitivity or hypersensitivity to potassium and/or vitamin C, or too much potassium and/or vitamin C in the diet. The disease-modifying drugs that are available and which the Applicant have tried over the years do not work. They modify the disease; they don't cure it. The effects of potassium and vitamin C on the body must be modified to relieve the symptoms of RA and related illnesses.

It appears that excess potassium inhibits kidney function, and causes sodium/potassium imbalance. The body then begins to lose fluids, which are needed by the body to regulate temperature, dilute acidity, digestion, etc. Inflammation is a result of the body's attempt to maintain homeostasis. The storage of fluids in the joints, soft tissue, etc. is what causes symptoms of RA and related illnesses. Excess fluid in the head may be the cause of migraines.

Build-up of acid in the body may be neutralized with calcium and vitamin D (for calcium absorption) or the body will retain fluids in an attempt to dilute said acid. Preferably, a greater amount of vitamin D than the recommended daily amount is needed.

It appears that what you drink also makes a difference. Many fruit juices have potassium and/or vitamin C in it. So cutting back on these fruit juices will generally not help as it appears that avoiding these fruit juices will provide a better result. It appears that as long as a drink has potassium and/or vitamin C, it will cause inflammation, especially if a large amount is consumed. It appears that drinking water, coffee, beer, and/or even sodas not flavored with fruit juices is what relieves the symptoms of RA. But even these beverages should be taken in moderation because they also disrupt the delicate potassium-sodium balance although not as much as the beverages with potassium. Some people are not willing to part with their morning orange juice or the healthy carrot, grape, or apple juices. And many people are now drinking the exotic acai, noni, and pomegranate juices. These may contain antioxidants but they also contain potassium and/or vitamin C. They appear to be a disaster for RA patients. It appears that potassium and/or vitamin C in the drinks will cause the kidneys to just about shut down. It appears that vitamin C is almost as bad as potassium.

It appears that the mineral magnesium does not cause the inflammation, but appears that it exacerbates the problem because magnesium increases circulation so that the fluid in the inflammation is moved around the body to different joints and to the muscles sometimes settling in the ankles and knees making it impossible to walk or settling in the soft tissue of the arms making it impossible to raise the arms to brush the teeth or comb the hair.

As a non-limiting aspect of the present invention, a treatment or cure for RA and related illnesses includes at least 1 of the following:

a) moderating, or discontinuing (if case is severe) intake of potassium and/or vitamin C.
This may also apply to caffeine and/or any other diuretics. Preferably, daily intake of each of potassium and vitamin C as part of a diet and/or a supplement is no more than about 100 mg, more preferably in the range of 0 mg to about 100 mg, and most preferably in the range of about 1 mg to about 90 mg;
b) fluid intake is preferably limited to water only until condition improves;
c) fluid/water is preferably administered about four (4) ounces every four hours.
Preferably, no more than about six (6) ounces per four hours;
d) fluid intake may preferably increase as condition improves;
e) increase in intake of an effective amount of sodium will speed recovery. Preferably, daily intake of sodium as part of a diet and/or a supplement is at least about 500 milligrams; and
f) if the individual's diet is usually bland or if daily caloric intake is low, an individual may preferably have to continue sodium supplement indefinitely.

Of secondary concern is calcium and/or vitamin D intake, as part of a diet and/or a supplement:

a) calcium, if taken as a supplement, is preferably taken in liquid or capsule form, but other forms should be effective also;
b) calcium, if taken as a supplement, is preferably taken as about 500 milligram doses twice per day, and preferably with meals;
c) vitamin D, if taken as a supplement, is preferably taken with calcium. If taken in capsule form, Vitamin D is preferably taken with the meal as with calcium. Or, if vitamin D is taken in the form of cod liver oil, it can be taken about a half hour or 30 minutes before meals;
d) vitamin D, if taken as a supplement, is preferably taken in doses of about one thousand (1,000) international units per day, for a minimum of three months;
e) after three months, vitamin D should be tapered to about 500 IU daily indefinitely; and
f) in winter months when there is less sunshine, or in the case of individuals who don't receive much sun exposure, vitamin D intake preferably remains at about 1,000 IU daily.

With treatment, the symptoms of RA and related diseases may be reduced by the second month and completely reversed and relieved in four months.

It is to be understood that the present invention is not limited to the embodiments and/or aspects described above, but encompasses any and all embodiments and/or aspects within the spirit of the invention.

1. A method for relieving symptoms of rheumatoid arthritis and related illnesses, the method comprising providing daily intake that comprises an amount of potassium ranging from no amount of potassium to about 100 mg of potassium to a subject in need whereby a reduction of the symptoms occurs, wherein the daily intake is at least one selected from the group consisting of a diet and a supplement.

2. The method for relieving symptoms of rheumatoid arthritis and related illnesses according to claim 1, wherein said amount of potassium ranging from 1 mg of potassium to about 90 mg of potassium.

3. The method for relieving symptoms of rheumatoid arthritis and related illnesses according to claim 1, wherein the daily intake further comprises an amount of calcium and an amount vitamin D, and wherein the amount of potassium is greater than no amount of potassium.

4. The method for relieving symptoms of rheumatoid arthritis and related illnesses according to claim 3, wherein the amount of vitamin D is greater than the recommended daily allowance of vitamin D.

5. The method for relieving symptoms of rheumatoid arthritis and related illnesses according to claim 1, wherein the daily intake further comprises an amount of vitamin C ranging from no amount of vitamin C to about 100 mg of vitamin C.

6. The method for relieving symptoms of rheumatoid arthritis and related illnesses according to claim 5, wherein said amount of vitamin C ranging from 1 mg of vitamin C to about 90 mg of vitamin C.

7. The method for relieving symptoms of rheumatoid arthritis and related illnesses according to claim 6, wherein the daily intake further comprises an amount of magnesium ranging from no amount of magnesium to a minimal amount of magnesium.

8. The method for relieving symptoms of rheumatoid arthritis and related illnesses according to claim 1, wherein the daily intake further comprises an amount of magnesium ranging from no amount of magnesium to a minimal amount of magnesium.

9. A method for relieving symptoms of rheumatoid arthritis and related illnesses, the method comprising providing a daily intake comprising an amount of vitamin C ranging from no amount of vitamin C to about 100 mg of vitamin C to a subject in need whereby a reduction of the symptoms occurs, wherein the daily intake is at least one selected from the group consisting of a diet and a supplement.

10. The method for relieving symptoms of rheumatoid arthritis and related illnesses according to claim 9, wherein said amount of vitamin C ranging from 1 mg of vitamin C to about 90 mg of vitamin C.

11. The method for relieving symptoms of rheumatoid arthritis and related illnesses according to claim 9, the daily intake further comprises an amount of calcium and an amount of vitamin D, and wherein the amount of vitamin C is greater than no amount of vitamin C.

12. The method for relieving symptoms of rheumatoid arthritis and related illnesses according to claim 11, wherein the amount of vitamin D is greater than the recommended daily allowance of vitamin D.

13. The method for relieving symptoms of rheumatoid arthritis and related illnesses according to claim 9, wherein the daily intake further comprises an amount of magnesium ranging from no amount of magnesium to a minimal amount of magnesium.

14. The method for relieving symptoms of rheumatoid arthritis and related illnesses according to claim 5, wherein the daily intake further comprises an amount of calcium and an amount vitamin D, and wherein at least one of the amount of potassium and the amount of vitamin C is greater than no amount of that substance.

15. The method for relieving symptoms of rheumatoid arthritis and related illnesses according to claim 14, wherein the amount of vitamin D is greater than the recommended daily allowance of vitamin D.

16. The method for relieving symptoms of rheumatoid arthritis and related illnesses according to claim 7, wherein the daily intake further comprises an amount of calcium and an amount vitamin D, and wherein at least one of the amount of potassium, amount of vitamin C, and amount of magnesium is greater than no amount of that substance.

17. The method for relieving symptoms of rheumatoid arthritis and related illnesses according to claim 16, wherein the amount of vitamin D is greater than the recommended daily allowance of vitamin D.

18. The method for relieving symptoms of rheumatoid arthritis and related illnesses according to claim 8, wherein the daily intake further comprises an amount of calcium and an amount vitamin D, and wherein at least one of the amount of potassium and the amount of magnesium is greater than no amount of that substance.

19. The method for relieving symptoms of rheumatoid arthritis and related illnesses according to claim 18, wherein the amount of vitamin D is greater than the recommended daily allowance of vitamin D.

20. The method for relieving symptoms of rheumatoid arthritis and related illnesses according to claim 9, wherein the daily intake further comprises an amount of calcium and an amount vitamin D, and wherein the amount of vitamin C is greater than no amount of vitamin C.

21. The method for relieving symptoms of rheumatoid arthritis and related illnesses according to claim 20, wherein the amount of vitamin D is greater than the recommended daily allowance of vitamin D.

22. The method for relieving symptoms of rheumatoid arthritis and related illnesses according to claim 13, wherein the daily intake further comprises an amount of calcium and an amount vitamin D, and wherein at least one of the amount of vitamin C and the amount of magnesium is greater than no amount of that substance.

23. The method for relieving symptoms of rheumatoid arthritis and related illnesses according to claim 22, wherein the amount of vitamin D is greater than the recommended daily allowance of vitamin D.

24. A composition for relieving symptoms of rheumatoid arthritis and related illnesses, said composition comprising an amount of calcium, an amount vitamin D, and at least one of potassium and vitamin C, wherein said composition is provided in a daily intake to a subject in need whereby a reduction of the symptoms occurs, wherein said daily intake is at least one selected from the group consisting of a diet and a supplement, wherein a first substance of said at least one of potassium and vitamin C has an amount ranging from about 1 mg of said first substance to about 100 mg of said first substance, and wherein a second substance of said at least one of potassium and vitamin C has an amount ranging from about 0 mg of said second substance to about 100 mg of said second substance.

25. The composition for relieving symptoms of rheumatoid arthritis and related illnesses according to claim 24, wherein said amount of vitamin D is greater than the recommended daily allowance of vitamin D.

26. The composition for relieving symptoms of rheumatoid arthritis and related illnesses according to claim 24, wherein said composition further comprises an amount of magnesium ranging from no amount of magnesium to a minimal amount of magnesium.
